(19) United States
(12) Patent Application Publication
(10) Pub. No.: US 2011/0044849 A1
(43) Pub. Date: Feb. 24, 2011

(54) DISINFECTING SPRAY DEVICE

(76) Inventors: Robert Infiesta, Weeki Wachee, FL (US); Sabrina Infiesta, Weeki Wachee, FL (US)

Correspondence Address: LAW OFFICE OF JERRY D. HAYNES , P.A, P.O. Box 350392, Fort Lauderdale, FL, 33335, US

(21) Appl. No.: 12/546,002

(22) Filed: Aug. 24, 2009

(51) Int. Cl. A61L 9/14; B65D 83/24

(52) U.S. Cl. 422/28; 222/402.14; 422/292

(57) ABSTRACT

The present invention relates to an aerosol spray assembly for disinfecting an enclosed area comprising: a aerosol can that contains a disinfectant; a spray nozzle on the top of the can that includes an activation tab; a locking slot that secures the activation tab in a closed position after the activation tab is depressed; and a 360-degree disinfectant spray that releases the disinfectant into the enclosed area. The disinfectant within the can may include an ammonium compound. The present invention disinfects an enclosed area by: placing the aerosol can in an upright position in the enclosed area; releasing a 360-degree disinfectant spray into the enclosed area by depressing an activation tab on the nozzle attached to the aerosol can; and locking the activation tab in a closed position to allow the total emission of disinfectant into the enclosed area.

### BACKGROUND OF THE INVENTION

1. Field of Invention

The present invention relates to a disinfecting spray device for disinfecting airborne bacteria and fungi within an enclosed space.

2. Description of Related Art

Eliminating and diminishing bacteria, viruses and other harmful microbes is a major concern for many households. Bacteria and viruses may cause sicknesses such as a cold, flu or irritation or exasperation of allergies. Various cleaning products are available to reduce or eliminate bacteria such as liquid or spray disinfectants. Liquid disinfectants are usually mixed with water and used to clean surfaces such as bathroom/kitchen counters, kitchen sink areas, walls and baseboards. This method of surface cleaning may be effective in reducing bacteria.

Airborne bacteria and viruses may be controlled through the use of spray, filters and air cleaning devices. Filters within a HVAC system may include webbing and chemical treatments that attempt to reduce the concentration of airborne bacteria. Specially designed filtered devices may be placed on a floor or a desktop that helps to filter and recirculate air being treated through the filter device. Furthermore sprays have been developed through the use of aerosol cans that emit disinfecting solutions into the atmosphere of a room. The emission of disinfectant spray is usually through a handheld aerosol can and is sprayed over a limited area for a short period of time and surface area.

The use of disinfectant spray may be more effective if a device is utilized to emit a consistent stream of evenly distributed disinfectant over an area of the enclosed space. Although some disinfectant sprays are available many are not effective in disinfecting a number of household viruses and bacteria such as influenza, rhinovirus, respiratory viruses, Herpes Simplex I and II, Hepatitis A or Poliovirus Type 1. An effective disinfectant spray needs to include the ability to kill a high percentage of bacteria, fungi and specifically to address any staph-related viruses that may be associated with staphylococcus aureus.

Therefore it would be advantageous to have a device available that may address the wide variety of household germs and bacteria. This is especially needed with respect to the recent spirit of specific flu viruses that may be of particular concern such as the swine flu virus.

### SUMMARY OF THE INVENTION

The present invention relates to an aerosol spray assembly for disinfecting an enclosed area comprising: an aerosol can, where the aerosol can contains a disinfectant; a spray nozzle, where the spray nozzle is on the top of the can and includes an activation tab; a locking slot, where the locking slot secures the activation tab in a closed position after the activation tab is depressed; and a 360-degree disinfectant spray, where the spray releases the disinfectant into the enclosed area. The disinfectant within the can may include an ammonium compound.

The present invention disinfects an enclosed area by: placing the aerosol can in an upright position in the enclosed area; releasing a 360-degree disinfectant spray, where the spray releases the disinfectant into the enclosed area by depressing an activation tab on a nozzle attached to the aerosol can; and locking the activation tab in a closed position to allow the total emission of disinfectant into the enclosed area. The spray is released in a fog-type emission and allows the entire contents of the aerosol can to be released.

### BRIEF DESCRIPTION OF DRAWINGS

FIG. 1 depicts a 360-degree aerosol-propelling assembly according to the present invention.

FIG. 2A depicts a top view of a spray cap associated with the aerosol assembly according to the present invention.

FIG. 2B depicts a bottom view of the spray cap according to the present invention.

FIG. 2C provides a prospective view of the spray cap according to the present invention.

### DETAILED DESCRIPTION

The present invention relates to a disinfecting spray that is utilized to eliminate airborne bacteria, viruses and mold spurs that may thrive in an indoor environment. The present invention utilizes an aerosol can that emits a disinfecting fog into an enclosed area and therefore disinfects the entire area exposed to the fog. The disinfectant spray according to the present invention can help to eliminate odors, eliminate bacteria and kills common household germs such as rhinovirus, influenza, salmonella, mold and mildew.

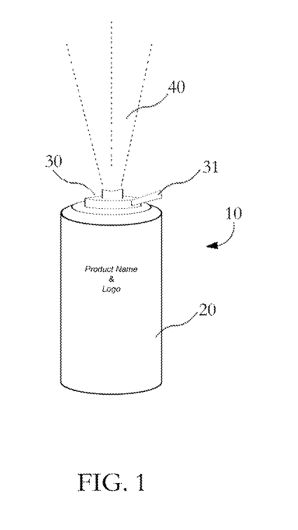

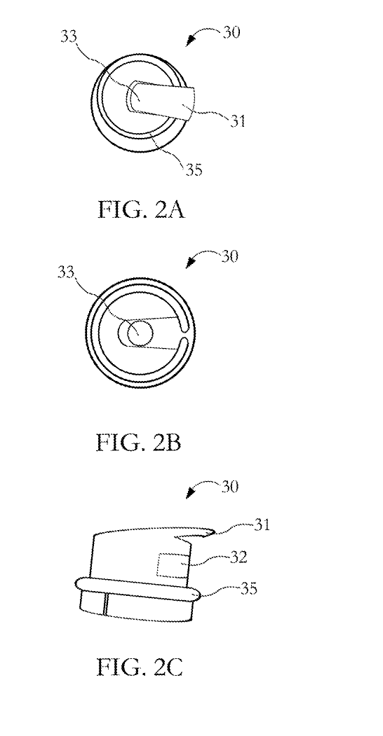

The present invention provides a disinfectant spray that propels a disinfecting compound into the air. FIG. 1 depicts an Aerosol Assembly 10 that includes an Aerosol Spray Can 20 with a Spray Nozzle 30 that has an Activation Tab 31 attached thereto. In order to activate the disinfectant spray, an individual depresses the Activation Tab 31 and places the tab into a Locking Slot 32 shown in FIG. 2C.

A Disinfecting Spray 40 emits a 360-degree spray dispersement that eliminates and kills germs, bacteria and odors upon contact. The spray emitted from the Aerosol Can 20 includes an ammonium compound selected from a group of alkyl dimethylbenzyl ammonium saccharinate in the amount of approximately 1 percent. The Disinfecting Spray 40 may also include ethanol for a quick-drying application of 75 percent of the total formula. The disinfectant spray according to the present invention is a single-use application that kills up to 99.9 percent of all germs existing in the air space where the aerosol is emitted.

The Spray Nozzle 30 includes a Spray Orifice 33 depicted in FIG. 2A which is larger than a normal size aerosol opening. This opening provides a means for the emission of the spray in a 360-degree fog-type emission. The Spray Nozzle 30 also includes a Sealing Lip 35 that is depicted in FIG. 2A where the Sealing Lip 35 provides a means to attach a cap or lid over the Spray Nozzle 30.

Other views of the Spray Nozzle 30 are provided in FIGS. 2B and 2C. A bottom view is depicted in FIG. 2B where the Spray Orifice 30 is clearly depicted with the Locking Slot 32 shown in FIG. 2C that provides a means to lock the Activation Tab 31 into a closed position. Once the Activation Tab 31 is closed, it opens the Orifice 33 and emits the emission of the disinfectant spray.

The Aerosol Assembly 10 according to the present invention provides a means to evenly distribute disinfectant spray throughout an enclosed area and thus mitigate odors, kill bacteria, eliminate fungi and destroy up to 99.9 percent of germs within the air. The Orifice 33 according to the present invention provides for vertical propulsion at a 360-degree angle therefore creating a fog type of a mist within the enclosed room where the disinfectant spray Assembly 10 is used. A child safety seal may be inserted within the Locking Slot 32 and therefore prevents the use of the disinfectant spray by children. Once this safety seal is released the Activation Tab 31 may be depressed and locked into the Locking Slot 32.

The aerosol assembly of the present invention provides a concentrated disinfectant to kill household germs and may be supplemented by scents or aromas to provide a fragrance once the disinfectant is emitted into the room. In addition to killing many household germs and viruses the disinfectant spray according to the present invention kills staph-type viruses on contact and therefore would be conducive for use in schools or locker rooms.

What is claimed is:

1. An aerosol spray assembly for disinfecting an enclosed area comprising:
a. an aerosol can, where the aerosol can contains a disinfectant;
b. a spray nozzle, where the spray nozzle is on the top of the can and includes an activation tab;
c. a locking slot, where the locking slot secures the activation tab in a closed position after the activation tab is depressed; and
d. a 360-degree disinfectant spray, where the spray releases the disinfectant into the enclosed area.

2. The aerosol spray assembly according to claim 1, where the disinfectant includes an ammonium compound.

3. The aerosol spray assembly according to claim 2, where the ammonium compound is selected from a group of alkyl dimethylbenzyl ammonium saccharinate in the amount of approximately 1 percent.

4. The aerosol spray assembly according to claim 1, where the disinfectant includes ethanol.

5. The aerosol spray assembly according to claim 1, where the spray releases a fog-type emission.

6. A method of disinfecting an enclosed area comprising:
a. placing an aerosol can in an upright position in an enclosed area, where the aerosol can contains a disinfectant;
b. releasing a 360-degree disinfectant spray, where the spray releases the disinfectant into the enclosed area by depressing an activation tab on a nozzle attached to the aerosol can; and
c. locking the activation tab in a closed position to allow the total emission of disinfectant into the enclosed area.

7. The method of disinfecting according to claim 6, where spray is released in a fog-type emission.

8. The method of disinfecting according to claim 6, where the step of releasing, allows the entire contents of the aerosol can to be released.
